(19) United States
(12) Patent Application Publication
(10) Pub. No.: US 2021/0069187 A1
(43) Pub. Date: Mar. 11, 2021

(54) Stable pharmaceutical composition of Selexipag

(71) Applicant: RK Pharma Solutions LLC, Piscataway, NJ (US)

(72) Inventors: Ravishanker Kovi, Monroe Township, NJ (US); Jayaraman Kannappan, Vadodara (IN); Raghu Kasu, Monmouth Jct, NJ (US); Thupalli Ajeykumar Reddy, Bangalore (IN); Devyani Dube, Vadodara (IN); Vamshi Yekkanti, Telangana (IN)

(21) Appl. No.: 16/893,869

(22) Filed: Jun. 5, 2020

(30) Foreign Application Priority Data
May 11, 2019 (IN) 201921018865

(51) Int. Cl. A61K 31/4965; A61K 47/10; A61K 47/36; A61K 9/20; A61K 47/38; A61K 47/12

(52) U.S. Cl. CPC A61K 31/4965; A61K 47/10; A61K 47/36; A61K 9/2077; A61K 47/38; A61K 9/2095; A61K 9/2054; A61K 9/2018; A61K 9/2059; A61K 9/2013; A61K 47/12

(57) ABSTRACT

A stable pharmaceutical composition of Selexipag is provided that includes a Selexipag-Mannitol pre mix and excipients. The present application relates to stable pharmaceutical compositions that include Selexipag which are suitable for oral administration. In particular, the application relates to pharmaceutical compositions of Selexipag in which acid impurity is less than 1% w/w. The application also relates to processes for making such compositions and use thereof in treating patients with pulmonary arterial hypertension.

### FIELD OF THE INVENTION

The present application relates to stable pharmaceutical compositions comprising Selexipag which are suitable for oral administration. In particular, the application relates to pharmaceutical compositions of Selexipag in which acid impurity is less than 1% w/w. The application also relates to processes for making such compositions and use thereof in treating patients with pulmonary arterial hypertension.

### BACKGROUND OF THE INVENTION

Selexipag, compound 1 chemically known as 2-{4-[N-(5,6-diphenylpyrazin-2-yl)-N-isopropylamino]butyloxy}-N-(methylsulfonyl)acetamide, has a molecular formula of C₂₆H₃₂N₄O₄S and a molecular weight of 496.62 g/mol. Selexipag has the following chemical structure as depicted in FIG. 1:

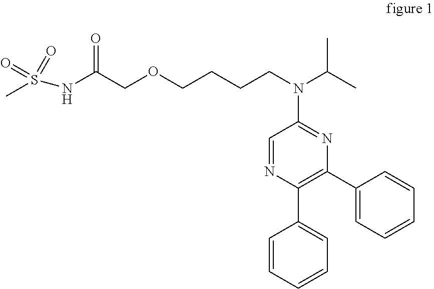 [SMILES: CC(C)N(CCCCOCC(=O)NS(C)(=O)=O)c1cnc(-c2ccccc2)c(-c2ccccc2)n1]

Selexipag is a pale-yellow crystalline powder that is practically insoluble in water. In the solid state Selexipag is very stable, is not hygroscopic, and is not light sensitive.

Depending on the dose strength, each round film-coated tablet for oral administration contains 200, 400, 600, 800, 1000, 1200, 1400, or 1600 mcg of Selexipag. The tablets include the following inactive ingredients: D-mannitol, corn starch, low substituted hydroxypropylcellulose, hydroxypropylcellulose, and magnesium stearate. The tablets are film coated with a coating material containing hypromellose, propylene glycol, titanium dioxide, carnauba wax along with mixtures of iron oxide red, iron oxide yellow or iron oxide black.

Selexipag has an excellent PGI2 receptor agonistic action, platelet aggregation inhibitory action, vasodilating action, bronchial muscle dilating action, lipid deposition inhibitory effect, leukocyte activation inhibitory action and the like.

U.S. Pat. No. 7,205,302, which is incorporated herein by reference, discusses Selexipag and its pharmaceutically acceptable salts. Processes to make Selexipag and pharmaceutically acceptable salts are also described, as are compositions containing Selexipag.

Prior art processes, however, leads to the formation of Selexipag acid impurity, which has the structure depicted in FIG. 2. Formed acid impurity is active metabolite in Selexipag, which is formed in the body.

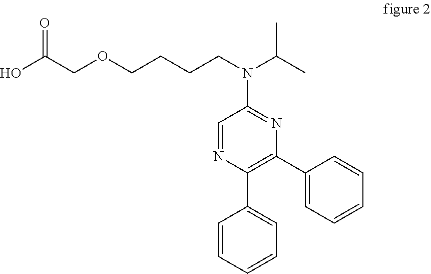 [SMILES: CC(C)N(CCCCOCC(=O)O)c1cnc(-c2ccccc2)c(-c2ccccc2)n1]

As per regulatory requirements, acid impurity should be controlled in formulations, so pharmaceutical manufacturers are continuously attempting to improve formulations to control (i.e., reduce) the impurity formation in stability, which ultimately helps to enhance and sustain their effects in human therapy. Correspondingly, there is a need for improve methods for improving the stability of such formulations.

Accordingly, at least one embodiment disclosed herein provides novel pharmaceutical composition having an effective amount of Selexipag, which retains its stability character upon storage, throughout its shelf-life.

### SUMMARY OF THE INVENTION

At least one aspect of the present application provides a stable pharmaceutical composition of Selexipag.

In one embodiment, a stable pharmaceutical composition of Selexipag is provided (combination of excipients and Selexipag Mannitol pre mix) having impurity less than 1% w/w.

In another embodiment, a process is provided for the preparation of the stable pharmaceutical composition of Selexipag having impurity less than 1% w/w.

In another embodiment, a stable pharmaceutical composition of Selexipag tablet is provided that is produced by mixing the dummy granules (combination of excipients, having water content (water by KF) less than 5%) with Selexipag Mannitol pre mix. Less than 1% of impurity in Selexipag composition is obtained by controlling the moisture content of the Selexipag-mannitol premix formulation between 0.2%-5% and preferably water content less than 3.5% w/w. The water content between 0.2%-5% of Selexipag-mannitol premix is controlled by forming a dummy granules (combination of excipients) having a water content less than 3.5%.

In another embodiment, the present application discloses a process for the preparation of Selexipag-mannitol premix, which is used for the formulation of stable Selexipag composition.

In another embodiment, the tablet(s) are then coated with respective coating material to yield the final drug product. The drug product may then be stored by using molecular sieve, which further helps to control the formation of acid impurity.

### BRIEF DESCRIPTION OF THE DRAWINGS

FIG. 1 is a flow diagram showing a process for the preparation of tablets according to at leas one embodiment disclosed herein.

### DETAILED DESCRIPTION OF THE INVENTION

At least one aspect of the present application provides a stabilized pharmaceutical composition of Selexipag having acid impurity less than 1% w/w. Less than 1% of acid impurity may be controlled or otherwise achieved by maintaining water content (by KF) of Selexipag-mannitol premix between 0.2%-5% w/w and preferably water content less than 3.5% w/w. The 0.2%-5% w/w water content of Selexipag-mannitol formulation may be controlled by forming a dummy granulation having water content less than 3.5% w/w. Here, “stable” means acid impurity less than 1% w/w with respect to a Selexipag composition(s).

Selexipag formulation according to at least one embodiment has inactive ingredients, such as D-mannitol, cornstarch, low substituted hydroxypropylcellulose, hydroxypropylcellulose, and/or magnesium stearate. The tablets may then be film coated with a coating material containing hypromellose, propylene glycol, titanium dioxide, carnauba wax along with mixtures of iron oxide red, iron oxide yellow or iron oxide black.

The acid impurity in such formulation is formed due to the presence of presence of moisture in the mentioned excipients which is favored by alkali medium. Accordingly, the aim of the present disclosure is to control (i.e., lower) the moisture in the formulation which ultimately leads to a lower acid impurity.

In certain embodiments, disclosed herein is pharmaceutical formulation comprising in a solid dosage form for oral delivery”

Selexipag between about 0.2 mg and about 1.6 mg of total weight:
between about 0.2 and about 4% of an active ingredient;
between about 30% and about 95% of one or more diluents;
between about 0.5% and about 15% of a water-soluble binder;
between about 0.5 and about 10% of a disintegrant; and between about 0.2 and about 2% of a lubricant.

In another embodiment, the tablets containing such formulations are then coated with respective coating material to get drug product. The drug product may then be stored by using molecular sieve which further helps to control the formation of acid impurity.

Suitable diluents for the formulations disclosed herein include, but are not limited to, mannitol, lactose monohydrate, microcrystalline cellulose, and corn starch

In some embodiments, the diluent for the formulations disclosed herein may include mannitol present in an amount of from 60% to 90% w/w. In other embodiments the mannitol is present in an amount of from 75% to 80% w/w.

In some embodiments, the diluent is cornstarch present in an amount of from 6% to 12% w/w. In another embodiment, the cornstarch is present in an amount of from 5% to 10% w/w.

In some embodiments, a suitable diluent is a combination of a mannitol and a starch (e.g., corn starch). In other embodiments, mannitol is present in an amount of from 60% to 90% w/w and cornstarch present in an amount of from 6% to 12% w/w. In other embodiments, mannitol is present in an amount of from 65% to 85% w/w and cornstarch present in an amount of from 6% to 12% w/w. In other embodiments, mannitol is present in an amount of from 70% to 80% w/w and cornstarch present in an amount of from 5% to 10% w/w.

Suitable binders for the formulations disclosed herein include, but are not limited to, gelatin, glucose, lactose, cellulose derivatives such as methyl cellulose, ethyl cellulose, hydroxypropylmethyl cellulose, hydroxypropyl cellulose, starch, polyvinylpyrrolidone(povidone), sodium alginate, carboxymethylcellulose, acacia etc.

In some embodiments, the binder is pharmaceutically acceptable hydroxypropyl cellulose. In some embodiments, hydroxypropyl cellulose demonstrates an increase in compressibility of the formulation, has good binding characteristics, and provides sufficient hardness to tablets formed by the methods disclosed herein

The use of hydroxypropyl cellulose as a binder enables the methods disclosed herein to form hard, strong tablets at low machine pressure, which reduces instances of chipping and capping of the resulting tablets as well as reducing the friability of the resulting tablets. The grade of hydroxypropyl cellulose chosen has been determined in the course of the evaluation of the formulations herein to contribute to improved, superior flow properties as well as to enhancing lubrication of the powders in the die cavity and on punch faces for forming indentations on the tablet surface such that the hydroxypropyl cellulose aids in preventing adherence of tablets to the punches during compression.

Suitable disintegrant for the formulations disclosed herein include, but are not limited to, povidone, croscarmellose sodium, sodium starch glycolate, 1-hydroxypropyl cellulose, and crospovidone

The formulations made in accordance with the methods disclosed herein may include at least one or more lubricant. The lubricants may be any suitable lubricants which contribute to the compressibility, flowability, and homogeneity of the formulation and which minimize segregation and do not significantly interfere with the slow release mechanism of the binders as set forth above.

In some embodiments, the lubricant functions to facilitate compression of the tablets and ejection of the tablets from the die cavity. Such lubricants may be hydrophilic or hydrophobic and can include magnesium stearate, Lubritab®, stearic acid, calcium stearate, and sodium stearyl fumarate, talc, and other lubricants known in the art or to be developed which exhibit acceptable or comparable properties.

Embodiment of the present invention will now be described more fully hereinafter with reference to the accompanying examples and experiments, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

In particular embodiments, the Selexipag that is incorporated into the dosage form is a mixture of Selexipag and mannitol. The Selexipag-mannitol mixture is preferably about 98% to about 100% pure.

In another aspect, methods are provided for making a various dosage form of Selexipag, pharmaceutically acceptable salts thereof, or pharmaceutically acceptable solvates thereof, delivering from 0.2 mg to 1.6 mg of Selexipag. In particular embodiments, the dosage form has 0.2 mg, 0.4 mg, 0.6 mg, 0.8 mg, 1 mg, 1.2 mg, 1.4 mg and 1.6 mg of Selexipag or Selexipag equivalent.

In one embodiment, the dosage form may be prepared by a process that includes the steps of:
a. weighing Selexipag mannitol premix; and
b. combining the Selexipag mannitol premix with dummy granules to form Selexipag formulation(s).

Within the scope of this embodiment, 0.2 mg to about 1.6 mg Selexipag may be weighed for combining with the dummy granules. In some embodiments, the mass ratio (w/w) of Selexipag to the dummy granules is from about 1:1 to about 1:35. In other embodiments the mass ratio is from about 1:10 to about 1:30.

As used herein, a formulation that includes at least Selexipag (or solvates thereof) and the dummy granules. In some embodiments, the method of preparing the Selexipag dosage form may further include steps of adding additional pharmaceutically acceptable excipients.

In one or more embodiments, the method further includes adding a binder, a diluent or mixtures thereof. In some embodiments, the binder, diluent and disintegrant are combined before adding to Selexipag mannitol premix. In other embodiments, the method may include adding a lubricant. In some embodiments, the lubricant is added to the extended release mixture containing Selexipag and the other excipients. In some embodiments, a binder, diluent, lubricant, and disintegrant are included in mixture, and said is a directly compressible formulation. Examples of suitable binders, diluents, lubricants, and disintegrants along with particularly useful properties of each are expressly discussed in detail above with regard to components of the Selexipag formulation.

In another embodiment of present invention a process for the preparation of Selexipag-mannitol premix is provided which is used for the formulation of stable Selexipag composition.

Selexipag is synthesized as per the process known in the prior art. Selexipag may then be mixed with other excipient to formulate Selexipag compositions.

As dosage of Selexipag in formulation is quite low to handle Selexipag-mannitol premix was prepared. Selexipag mannitol premix is prepared by following the process discussed below.

Selexipag may be dissolved in a suitable solvent to get the clear solution. If slurry is obtained then the reaction is heated to get the clear solution. The clear solution may then be filtered through micron paper to remove unwanted foreign particulate matters. The filtered solution may then be distilled under vacuum to get a reduced volume. The solution may then be cooled to get to room temperature. D-Mannitol may then be added to the above solution and stirred for few hours at 10-15° C. Finally, the reaction mass may be fildered and washed with suitable solvent to get a Selexipag-mannitol premix.

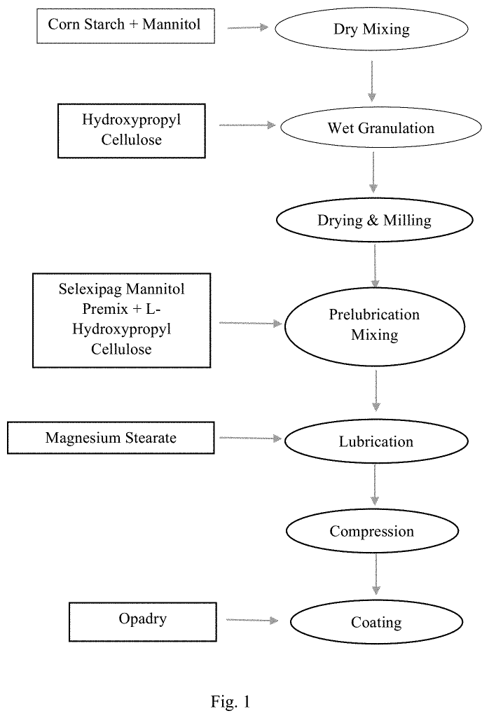

In particularly useful embodiments, tablets may then be prepared by methods disclosed herein. In one embodiment, tablets may be prepared by a process included in FIG. 1.

Within the scope of this embodiment, the lubricant, binder, diluent and disintegrant may be any lubricant, binder, diluent and disintegrant as disclosed above. In particularly useful embodiments, the diluent is cornstarch and mannitol, the binder is hydroxyproylcellulose, disintegrant is L-hydroxypropyl cellulose and lubricant is magnesium stearate.

Thereafter, the tablet (sometimes referred to as the core) may be film coated with a moisture barrier coating or color coating such as Opadry Purple, Opadry Blue, or Opadry White for identification, taste masking, and appearance purposes to provide a film-coated tablet. The film coating does not substantially affect the release rate of the Selexipag from the tablet, since the coating rapidly dissolves in the stomach. The outer film coating may be from between 0.03 mm to 1.0 mm in thickness.

The coated tablet may be then stored in 40 cc HDPE bottle with CR closure containing 2 g molecular sieve.

### EXAMPLES AND EXPERIMENTS

Process for the preparation of Selexipag mannitol premix.
1. Selexipag is dissolved in n-butyl acetate at room temperature (RT).
2. Reaction mass is then heated to get 80° C. to get clear reaction mass.
3. Reaction mixture is then filtered through micron filter.
4. Filtered mixture is then distilled under vacuum to get reduced volume of solution.
5. Solution is cooled to get 25-30° C.
6. D-Mannitol is then added to above solution and stirred for about 10 hours at 10-15° C.
7. Filter the reaction mass and wash with isopropyl acetate to get Selexipag-mannitol premix.

### Examples 1, 2, and 3

Pharmaceutical composition containing example 1 (0.2 mg of Selexipag), and example 2 and 3 (1.6 mg of Selexipag) were formulated as per process depicted below

Experiment:

### Manufacturing Procedure:

a. Sifting cornstarch and mannitol through ss #40 mesh ATM.
b. Dry Mixing:
   1. cornstarch and mannitol were cosifted through ss #40 mesh ASTM.
   2. step 1 was blended in V-blender for 15 minutes.
c. Binder solution preparation: 20% w/w solution of hydroxypropyl Cellulose was prepared in water.
d. Mix above solution.
e. Granulation: Granulation was done of above mixture.
f. Drying: Granules were dried for 15 minutes at 60° C. at 70 air flow.
g. Sizing: Granules were sifted through ss #30 mesh ASTM targeting 40% retention on ss #60 mesh ASTM and 60% fines.
h. Pre-lubrication Sifting:
   1. Selexipag mannitol premix was cosifted with equal quantity of fines through ss #60 mesh ASTM.
   2. Step 1 was cosifted with equal quantity of fines through ss #60 mesh ASTM.
   3. Step 2 was repeated till complete geometrical dilution with fines and then with granules.
   4. l-hydroxypropyl cellulose was sifted through ss #40 mesh ASTM and then added to step 3.
   5. Step 4 was blended for 15 minutes in V blender at 15-20 rpm.
i. Lubrication Sifting: Magnesium stearate was sifted through ss #60 mesh ASTM
j. Lubrication Mixing: step h was added to step i and blended for 5 minutes.
k. Tablets were compressed keeping tablet weight of 100 mg.
l. Core tablets were coated using Opadry 10% w/w solution in water up to 3% built up.
m. Storage of coated tablet in molecular sieve.

Tables:

Table 1 sets forth formulations prepared in accordance with the above-described methods.
Table 2 set forth the analytical profiles for Examples 1, 2, and 3.

TABLE 1
Selexipag Compositions for examples 1, 2 and 3

| | | Example 1 (0-2 mg) | | Example 2 (1.6 mg) | | Example 3 (1.6 mg) | |
|---|---|---|---|---|---|---|---|
| S. No | Ingredients | % w/w | mg/tab | % w/w | mg/tab | % w/w | mg/tab |
| 1 | Corn Starch | 9.71 | 10 | 9.71 | 10 | 9.71 | 10 |
| 2 | Mannitol | 76.87 | 79.2 | 74.00 | 76.4 | 74.00 | 76.4 |
| 3 | Hydroxypropyl Cellulose | 5.83 | 6 | 5.83 | 6 | 5.83 | 6 |
| 4 | Water | qs | qs | qs | qs | qs | qs |
| 5 | Selexipag Mannitol premix | 0.41 | 0.4 | 3.28 | 3.2 | 3.28 | 3.2 |
| 6 | L- Hydroxypropyl Cellulose | 3.88 | 4 | 3.88 | 4 | 3.88 | 4 |
| 7 | Magnesium stearate | 0.39 | 0.4 | 0.39 | 0.4 | 0.39 | 0.4 |
| 8 | Opadry | 2.91 | 3 | 2.91 | 3 | 2.91 | 3 |
| | Total | 100 | 103 | 100 | 103 | 100 | 103 |

TABLE 2
Analytical profiles for examples 1, 2 and 3

| | Example 1 (0.2 mg) | | Example 2 & 3 (1.6 mg) | |
|---|---|---|---|---|
| | Innovator | | Innovator | |
| | Initial | 1 M 40/75 | Initial | 2 M 40/75 |
| Assay | 95.4 | 95.9 | 96.3 | 95.7 |
| Water content | 3.77 | 3.91 | 2.81 | 2.65 |
| RRT 0.88 (Acid imp) | 0.05 | 1.31 | 0.03 | 0.49 |
| RRT 1.31 | | 0.47 | | 0.16 |
| Highest unknown imp | 0.04 | 0.47 | 0.03 | 0.16 |
| Total Imp | 0.11 | 1.92 | 0.07 | 0.71 |

Example 1:

| | Example 1 (0.2 mg) | | | | | |
|---|---|---|---|---|---|---|
| | | Dummy granules | | | | |
| | | 20 D | 3 M | 6 M | 3 M | 6 M |
| | Initial | 40/75 | 40/75 | 40/75 | 25/60 | 25/60 |
| Assay | 99.8 | NA | NA | 100.3 | NA | 101 |
| Water content | 1.2 | 0.73 | 1.36 | 100.3 | NA | 101 |
| RRT 0.88 (Acid imp) | 0.04 | 0.04 | 0.02 | 0.08 | 0.03 | 0.05 |
| Isopropylamine | ND | 0.01 | 0.01 | ND | ND | ND |
| RRT 1.31 | ND | ND | 0.07 | 0.33 | 0.02 | 0.07 |
| highest unknown imp | 0.02 | 0.01 | 0.07 | 0.33 | 0.02 | 0.07 |
| total imp | 0.08 | 0.07 | 0.11 | 0.49 | 0.07 | 0.2 |

Example 2:

| | Example 2 (1.6 mg) | | | | | |
|---|---|---|---|---|---|---|
| | | Dummy granules | | | | |
| | | 1 M | 3 M | 6 M | 3 M | 6 M |
| | Initial | 40/75 | 40/75 | 40/75 | 25/60 | 25/60 |
| Assay | 95.1 | 96.1 | 94.4 | 96.1 | 95.8 | 96.8 |
| Water content | 1.3 | 1.61 | 1.6 | 1.06 | 1.64 | 0.8 |
| RRT 0.88 (Acid imp) | 0.04 | 0.04 | 0.03 | 0.06 | 0.04 | 0.05 |
| Isopropylamine | ND | ND | ND | 0.02 | ND | 0.02 |
| RRT 1.31 | ND | ND | 0.04 | 0.1 | ND | 0.02 |
| highest unknown imp | 0.38 | 0.05 | 0.04 | 0.1 | 0.01 | 0.02 |
| total imp | 0.89 | 0.19 | 0.08 | 0.21 | 0.05 | 0.12 |

Example 3:

| | Example 3 (1.6 mg) | | | |
|---|---|---|---|---|
| | | Dummy granules | | |
| | | 1 M | 2 M | 6 M |
| | Initial | 40/75 | 40/75 | 40/75 |
| Assay | 106.6 | 101.8 | 103.3 | 104.5 |
| Water content | 1.36 | 1.16 | 1.55 | 0.55 |
| RRT 0.88 (Acid imp) | 0.04 | 0.04 | 0.04 | 0.05 |
| Isopropylamine | ND | ND | ND | 0.01 |
| RRT 1.31 | ND | ND | 0.06 | 0.1 |
| highest unknown imp | 0.01 | 0.02 | 0.06 | 0.1 |
| total imp | 0.06 | 0.06 | 0.12 | 0.18 |

Although the compositions, schemes and methods of the present disclosure have been described with reference to exemplary embodiments thereof, the present disclosure is not limited thereby. Indeed, the exemplary embodiments are implementations of the disclosed methods are provided for illustrative and non-limitative purposes. Changes, modifications, enhancements and/or refinements to the disclosed methods may be made without departing from the spirit or scope of the present disclosure. Accordingly, such changes, modifications, enhancements and/or refinements are encompassed within the scope of the present invention.

1. A stable pharmaceutical composition, comprising:
a Selexipag-Mannitol pre mix;
one or more diluents;
a water-soluble binder;
a disintegrant; and
a lubricant.

2. The stable pharmaceutical composition of claim 1, wherein the Selexipag composition has impurity less than 1% w/w.

3. The stable pharmaceutical composition of claim 1, comprising:
a) 0.2 to 4% w/w of an Selexipag-Mannitol pre mix;
b) 30% to 95% w/w of one or more diluents;
c) 0.5% to 15% w/w of water-soluble binder;
d) 0.5 to 10% w/w of disintegrant; and
e) 0.2 to 2% w/w of lubricant.

4. The stable pharmaceutical composition of claim 3, wherein Selexipag is present between about 0.2 mg and about 1.6 mg of total weight.

5. The stable pharmaceutical composition of claim 1, wherein diluents are selected form the group consisting of mannitol, lactose monohydrate, and microcrystalline cellulose, and corn starch.

6. The stable pharmaceutical composition of claim 1 wherein, the diluent is mannitol present in an amount from 60% to 90% w/w.

7. The stable pharmaceutical composition of claim 1 wherein, the diluent is mannitol present in an amount from 75% to 80% w/w.

8. The stable pharmaceutical composition of claim 1, wherein the diluent is cornstarch present in an amount from 6% to 12% w/w.

9. The stable pharmaceutical composition of claim 1, wherein the diluent is cornstarch present in an amount from 5% to 10% w/w.

10. The stable pharmaceutical composition of claim 1, wherein the diluent is a combination of a mannitol and cornstarch, wherein mannitol is present in an amount from 60% to 90% w/w and cornstarch present in an amount from 6% to 12% w/w.

11. The stable pharmaceutical composition of claim 10, wherein mannitol is present in an amount from 70% to 80% w/w and cornstarch is present in an amount from 5% to 10% w/w.

12. The stable pharmaceutical composition of claim 1, wherein binders are selected from the group consisting of: gelatin, glucose, lactose, cellulose derivatives such as methyl cellulose, ethyl cellulose, hydroxypropylmethyl cellulose, hydroxypropyl cellulose, starch, polyvinylpyrrolidone (povidone), sodium alginate, 5 carboxymethylcellulose, and acacia.

13. The stable pharmaceutical composition of claim 1, wherein the disintegrant is selected from the group consisting of: povidone, croscarmellose sodium, sodium starch glycolate, 1-hydroxypropyl 10 cellulose, and crospovidone.

14. The stable pharmaceutical composition of claim 1, wherein the lubricant is selected from the group consisting of: magnesium stearate, Lubritab, stearic acid, calcium stearate, and sodium stearyl fumarate, and talc.

15. The stable pharmaceutical composition of claim 1, wherein the pharmaceutical composition is in the form of a tablet that comprises the dummy granules mixed with the Selexipag-Mannitol pre mix.

16. The stable pharmaceutical composition of claim 15, wherein the dummy granules comprise a combination of excipients having water content (water by KF) less than 5%.

17. The stable pharmaceutical composition of claim 1, wherein Selexipag impurity is less than 1% and moisture content of the Selexipag-mannitol premix formulation is between 0.2%-5% w/w.

18. The stable pharmaceutical composition of claim 17, wherein moisture content of the Selexipag-mannitol premix formulation is less than 3.5% w/w.

19. A method for producing the Selexipag-Mannitol pre mix of claim 1, comprising:
a. dissolving Selexipag in a suitable solvent to get the clear solution;
b. filtering the clear solution;
c. distilling the filtered solution under vacuum to get reduced volume;
d. cooling the solution to room temperature;
e. adding D-Mannitol to the solution and stirred for few hours at 10-15° C.; and
f. filtering and washing the reaction mass with suitable solvent to get the Selexipag-mannitol premix.
